(19) United States
(12) Patent Application Publication
(10) Pub. No.: US 2026/0260352 A1
(43) Pub. Date: Sep. 3, 2026

(54) SYSTEMS AND METHODS FOR INDIVIDUALIZED ESTIMATION OF BASELINE DIAGNOSTIC MAPS AND PROFILES

(71) Applicant: Oregon Health & Science University, Portland, OR (US)

(72) Inventors: Ou Tan, Portland, OR (US); David Huang, Portland, OR (US)

(21) Appl. No.: 19/555,682

(22) Filed: Mar. 3, 2026

Related U.S. Application Data
(60) Provisional application No. 63/765,779, filed on Mar. 3, 2025.

(51) Int. Cl. G06T 7/00; A61B 3/10; A61B 3/12

(52) U.S. Cl. CPC G06T 7/0014; A61B 3/102; A61B 3/1241; G06T 2207/10101; G06T 2207/20081; G06T 2207/30041

(57) ABSTRACT

Embodiments herein relate to diagnosis of eye diseases by estimating individualized baseline Optical Coherence Tomography (OCT) or OCT angiography (OCTA) characteristics of the eye using deep learning that takes into account individual ocular characteristics. A difference between the baseline characteristics and measured characteristics indicates whether disease is present. An example implementation uses nerve fiber layer thickness (NFLT) to detect glaucoma. In a set of healthy eyes, circumpapillary NFLT profiles and vascular patterns were measured using OCT. Generative deep learning AI models were trained to reconstruct the individualized baseline NFLT, a customized normal reference based on each eye's own vascular pattern, axial length (AL) and spherical equivalent (SE) refractive error, disc size, and individual demography information. Two deep learning models were developed. A first (MAG) model used actual AL and SE, while a second (REG) model estimated AL and SE using vascular pattern as input.

### CROSS-REFERENCE TO RELATED APPLICATIONS

This application claims the benefit of U.S. provisional patent application No. 63/765,779, filed Mar. 3, 2025, titled “Systems And Methods For Individualized Estimation Of Baseline Glaucoma Diagnostic Maps And Profiles,” which is incorporated herein by reference.

### ACKNOWLEDGEMENT OF GOVERNMENT SUPPORT

This invention was made with government support under R01 EY023285 and R21 EY032146 awarded by The National Institutes of Health. The government has certain rights in the invention.

### TECHNICAL FIELD

This disclosure relates to systems and methods for estimating individualized baseline maps and profiles of various parameters relevant for diagnosis and management of glaucoma and other eye diseases.

### BACKGROUND

Various techniques have been developed to test for eye diseases using Optical Coherence Tomography (OCT). For example, glaucoma is associated with the progressive loss of retinal nerve fiber layer thickness (NFLT). OCT can be used to detect and monitor nerve fiber layer thinning. Detection of pathological thinning is based on the premise that the NFLT measured in an individual person can be compared to a theoretical baseline value prior to disease onset. In some cases (i.e., the glaucoma diagnostic packages offered by OCT manufacturers), NFLT distribution in a healthy population is used as a generic baseline. A normative database with hundreds of healthy eyes can be collected for a specific OCT model in the Food & Drug Administration (FDA) clearance process. NFLT variation associated with demographic (age, race, sex) factors is accounted for by regression or matching with the patient to be tested. However, this approach neglects significant variations in the NFLT associated with the vascular pattern and transverse optical magnification of the individual eye. Neglecting these individual variations can lead to false-positive diagnoses or false-negative misdiagnoses of glaucoma and other optic neuropathies.

Another problem of this approach is the false positive diagnosis of glaucoma in highly myopic eyes, which have thinner measured NFLT globally (and in most sectors) due to higher axial length (AL) and lower transverse optical magnification. The lower magnification causes the OCT scan pattern to span a larger area around the optic nerve head (ONH), and the NFLT decreases with greater distance from the ONH in a roughly reciprocal fashion. This problem is referred to as “red disease” because values below the 1 percentile cutoff of the healthy population are typically printed in red on OCT displays.

The solutions herein address the above and other issues by providing improved techniques to test for eye diseases using Optical Coherence Tomography (OCT).

### SUMMARY

Disclosed herein are systems and methods suitable to generate, from OCT data, individualized baseline profiles and maps of anatomical structures of the eye. In some embodiments, a deep learning generative model based on a conditional variational autoencoder (CVAE) architecture may be configured to receive as input patient-specific anatomic and non-anatomic characteristics, and generate as output an individualized baseline profile or map. In particular embodiments, a convolutional neural network for regression analysis (rCNN) may also be used to estimate certain individual features, such as AL and SE, from OCT data. These estimated individual features may themselves be used as inputs to a deep learning model for generating individualized baseline profiles and maps, thereby obviating the need to measure these features using other non-OCT ophthalmic devices or techniques.

In an example implementation, a computer-implemented method comprises measuring a diagnostic Optical Coherence Tomography (OCT) or OCT angiography (OCTA) characteristic of a patient's eye; calculating an individualized baseline of the diagnostic OCT or OCTA characteristic; calculating a difference between the individualized baseline of the OCT or OCTA characteristic and the measured OCT or OCTA characteristic; and determining whether the difference is consistent with a disease of the eye.

Additional aspects and advantages will be apparent from the following detailed description of preferred embodiments.

### BRIEF DESCRIPTION OF THE DRAWINGS

To easily identify the discussion of any particular element or act, the most significant digit or digits in a reference number refer to the figure number in which that element is first introduced.

FIG. 1A depicts an example generalized individualized baseline model 100, in accordance with various embodiments.

FIG. 1B depicts example eye anatomy maps as inputs to the model 100 of FIG. 1A, in accordance with various embodiments.

FIG. 1C depicts a table of example inputs and outputs of the model 100, in accordance with various embodiments.

FIG. 2 depicts a set of example eye anatomy maps for use in the model 100 of FIG. 1A, in accordance with various embodiments.

FIG. 3A depicts a table of characteristics of study subjects from two cohorts, in accordance with various embodiments.

FIG. 3B depicts a table of associations between nerve fiber layer thickness (NFLT) and predictive factors from the Hong Kong FAMILY cohort of FIG. 3A, in accordance with various embodiments.

FIG. 3C depicts a table of prediction errors in μm of NFLT in healthy eyes in the two cohorts of FIG. 3A, in accordance with various embodiments.

FIG. 4 depicts prediction error profiles (plots 400) and significance masks (plots 450) for p<0.05 compared to a population average over five models, for the Hong Kong FAMILY cohort of FIG. 3A, in accordance with various embodiments.

FIG. 5 depicts present prediction error profiles (plots 500) over five models, for the Casey Eye Institute (CEI) cohort of FIG. 3A, in accordance with various embodiments.

FIG. 6A depicts a table of false positive rate based on a 5-percentile cutoff estimated from emmetropia eyes for the Hong Kong FAMILY cohort of FIG. 3A, in accordance with various embodiments.

FIG. 6B depicts a table of differences between a normal reference mean and a 5-percentile cut-point for the Hong Kong FAMILY cohort of FIG. 3A, in accordance with various embodiments.

FIG. 7 depicts a table of results from testing a nerve fiber layer thickness individualized baseline model with an Advanced Image for Glaucoma (AIG) study dataset, in accordance with various embodiments. FIG. 8A presents a circular pattern of 13 rings covering a peripapillary area around the optic disc as utilized in ONH scans, in accordance with various embodiments.

FIG. 8B presents a representative OCT B-scan with NFLT boundaries (solid lines 810 and 811) and vessel shadow detected (dots 820-831), in accordance with various embodiments.

FIG. 8C presents a representative NFLT map and main vessel location map obtained from vessel shadow (example solid radial lines 840-847), where the center area masked the optic disc, in accordance with various embodiments.

FIG. 8D presents a representative NFLT (RNFLT) profile (plot 860) and vascular pattern (sets of circles 861-868) that were resampled on the re-centered 3.4 mm circle (circular dashed line 850 in FIG. 8C), in accordance with various embodiments.

FIG. 9 represents an overall structure of an example BASE model 900 in the training stage, consistent with FIG. 1A, in accordance with various embodiments.

FIG. 10 represents example BASE model conditions including age, gender and signal strength index (SSI) conditions, in an example implementation of the conditions 990 of FIG. 9, in accordance with various embodiments.

FIG. 11 represents example MAG model conditions, including the conditions listed in the BASE model of FIG. 10, plus disc area, measured axial length (AL) and spherical equivalent error (SE), in another example implementation of the conditions 990 of FIG. 9, in accordance with various embodiments.

FIG. 12 depicts an example REG model 1200 used to provide REG model conditions 1250, in another example implementation of the conditions 990 of FIG. 9, in accordance with various embodiments.

FIG. 13 represents the overall structure of another example deep learning model 1300 in the generating stage, consistent with FIG. 1A, in accordance with various embodiments.

FIG. 14 depicts a block diagram of an example system 1400 for estimation of individualized baseline diagnostic maps and profiles, in accordance with various embodiments.

FIG. 15 depicts a block diagram of an example computing system, in accordance with various embodiments.

FIG. 16 depicts a flowchart of an example process for predicting a presence of disease in a patient's eye, in accordance with various embodiments.

### DETAILED DESCRIPTION OF EMBODIMENTS

Optical coherence tomography (OCT) measurements can be used for detecting diseases of the eye. In one possible example implementation, retinal nerve fiber layer thickness (NFLT) profiles or maps can be used for glaucoma diagnosis. Glaucoma damage is detected by comparing the individual's NFLT pattern against a pre-disease baseline reference pattern. Since the pre-disease baseline of an individual is not available at the first visit to a doctor, the standard practice is to use the NFLT profile or map averaged from a healthy population as an estimate of the baseline reference. However, the NFLT distribution in the healthy population has a wide range of variations in axial eye length, refractive error, and retinal vascular pattern. The axial eye length and refractive error, in particular, affect the transverse optical magnification of the OCT scan and greatly impact the measured NFLT distribution. Further, each individual eye has anatomic variations that affect both the retinal vascular pattern and NFLT distribution, which are correlated with each other. Accounting for these individual variations could improve the estimation (also called “prediction”) of the baseline NFLT distribution to generate an individualized baseline reference customized to an individual eye and thereby improve the accuracy of glaucoma diagnosis by helping doctors distinguish glaucoma damage from normal inter-individual variations in NFLT distribution.

Disclosed herein are systems and methods for a generative artificial intelligence (AI) model to generate an individualized baseline NFLT distribution based on patient-specific anatomic and non-anatomic features. These characteristics may include, in some embodiments, the retinal vascular pattern, axial eye length, and refractive error of the specific eye, as well as the demographic information of the individual person. The AI method underlying the systems and methods described herein can include a variational autoencoder, a type of deep learning technique, in an example implementation.

Also described herein is an AI model to estimate the axial eye length and refractive error of the eye based on the retinal elevation and vascular pattern maps. The AI model can use a convolutional neural network for regression (rCNN). Since the NFLT, retinal elevation, and retinal vascular pattern are all obtained from OCT scans, the OCT system is the only instrumentation needed in the clinical application of the disclosed methods.

In an example application described below, a pilot study was conducted in a group of normal subjects that showed the generative AI-derived individualized baseline reference produced by the disclosed methods significantly reduced the prediction error for the individuals' NFLT compared to the prediction error of a generic reference based on the simple population average. The AI-derived individualized baseline also outperformed linear regression-based individualized baseline in terms of prediction error.

Also described herein is a generative AI method to create an individualized baseline NFLT distribution for each eye that accounts for magnification bias associated with the individual's axial eye length and refractive error, as well as retinal anatomic variation associated with the individual's retinal vascular pattern. Currently, in commercial OCT, the normal reference is adjusted only for demographic factors such as age and sex. The generative AI methods disclosed herein thus have the potential to provide much more accurate and individualized baseline to differentiate glaucoma damage or other eye disease from normal variations and thereby improve the accuracy of diagnosis.

An aspect of the disclosed generative AI model is that it can be trained using only data from normal eyes. This is advantageous because it is much easier to collect data from a large population of normal eyes, compared to the collection of data from glaucoma patients that require careful characterization by specialized clinicians and tests. This represents a substantial advantage of the disclosed systems and methods compared to other AI models for glaucoma diagnosis that requires data from both glaucomatous and normal eyes for the purpose of model training.

In some embodiments, the disclosed systems and methods may utilize OCT-derived retinal elevation and vascular pattern maps to estimate or predict axial eye length and refractive error, parameters that together specify the transverse optical magnification of an eye. By using information from the OCT scan itself to estimate and correct its own magnification bias, no other instruments are needed. This greatly improves the clinical practicality of the disclosed methods.

In further embodiments, the disclosed systems and methods described for the generation of individualized baseline for NFLT can be also extended to other OCT and OCT angiography measurements that are also affected by transverse optical magnification and anatomic variations. In the optic disc region these measurements include the disc rim width and cup-to-disc ratio from OCT scans, as well as retinal nerve fiber layer plexus capillary density from OCT angiography. In the macular regions, these measurements include the ganglion cell complex thickness and ganglion cell-inner plexiform layer thickness maps based on OCT scans, as well as superficial vascular complex vessel density and ganglion cell layer plexus vessel density from OCT angiography.

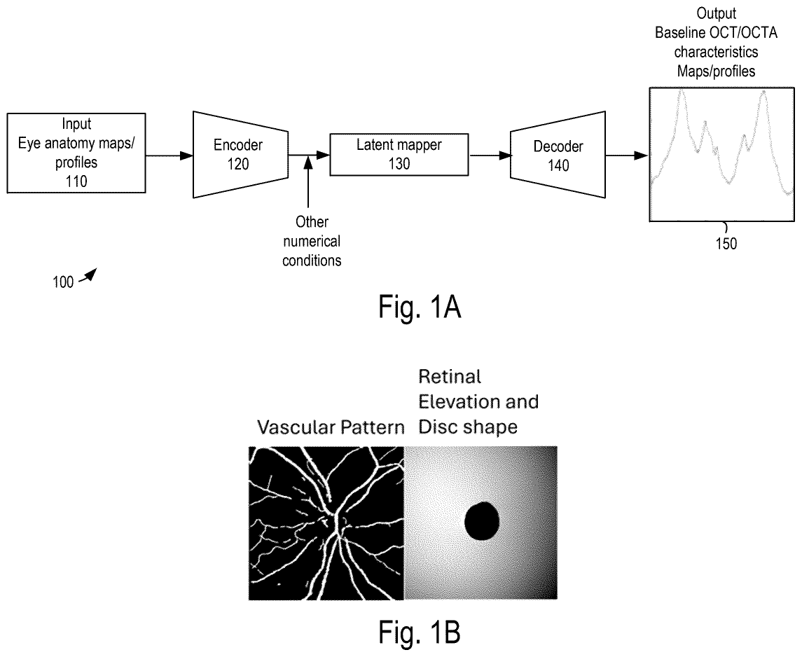

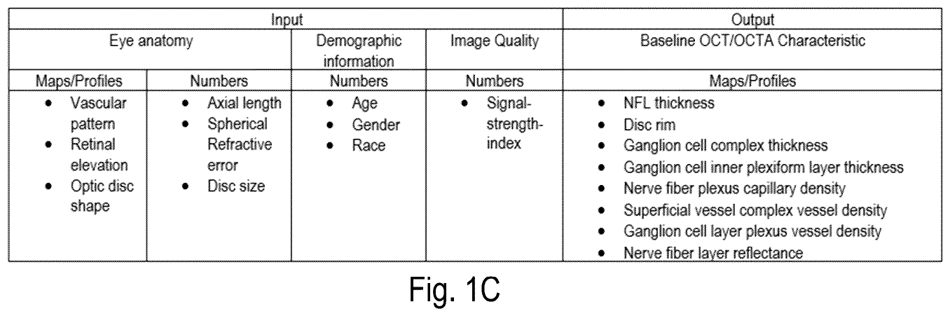

FIG. 1A depicts an example generalized individualized baseline model 100, in accordance with various embodiments.

A generative AI model, CVAE, is proposed to create a customized normal reference, or individualized baseline, using anatomic features and demographic information. Example testing involved glaucoma diagnosis. The AI model was trained on the Hong Kong Family dataset and validated on the Casey Eye Institute (CEI) and Advanced Image for Glaucoma (AIG) datasets. The generative model is only trained on normal eyes and can only generate an individualized baseline for a given scan of a specific eye, in an example implementation. For glaucoma diagnosis, the difference between the predicted NFL thickness individualized baseline matching the vascular pattern and the detected NFL thickness are used to test if the eye has glaucoma damage.

The above generative AI model can be generalized to input the eye anatomy, demographic information, and OCT image quality, and output the individual baseline of the OCT/OCT angiography map or profile. Therefore, a generalized individualized baseline model for diagnosis of a disease is proposed using any OCT or OCT angiography maps and profiles. The disease can include ocular and neurological diseases, such as glaucoma, other optic neuropathies, and multiple sclerosis. In example implementations, the generalization may include: 1) the deep learning model diagram; 2) the OCT/OCTA parameter used for diagnosis; 3) the dataset used for training.

The system includes one or more inputs 110, an encoder network 120, a latent mapping network 130, a decoder network 140, and one or more outputs 150. The inputs 110 may include eye anatomic feature maps or profiles derived from optical coherence tomography (OCT), including OCT angiography (OCTA), and optionally additional subject-specific information. The encoder network 120 is configured to transform the anatomical maps or profiles into a latent representation residing in a first latent representation space (source latent space), wherein the encoder generates one-dimensional (1D) or two-dimensional (2D) feature embeddings. Additional subject-specific features, including demographic or biometric information, may be converted into numerical form and concatenated with the encoder-generated latent representation, such numerical features serving as conditioning variables that, together with the encoder output, define the source latent representation provided to the latent mapping network. The latent mapping network 130 is configured to transform the source latent representation into a mapped latent representation residing in a second latent representation space (target latent space), thereby learning a transformation between latent manifolds and projecting the encoded features into a latent space corresponding to normative ocular characteristics. The decoder network 140 is configured to reconstruct one or more baseline OCT characteristics, including OCT or OCTA maps or profiles, from the mapped latent representation in the target latent space. The encoder network 120, latent mapping network 130, and decoder network 140 are each implemented as deep learning models and, in certain embodiments, are trained using OCT and eye anatomy data obtained exclusively from normal eyes, such that the learned latent representation spaces encode prior knowledge corresponding to normative ocular structure and the decoder reconstructs baseline characteristics representative of a healthy eye.

The inputs 110 are intended to include maps or profiles of eye anatomic features which are not sensitive to damage from a given disease of interest (i.e., the disease to be diagnosed, such as glaucoma). Similarly, the conditions to be concatenated to the output of encoder 120 whose numerical features represent eye anatomy, demography, and image quality are also insensitive to the structural changes or damage caused by the disease of interest. The output 150 from decoder 140 is in the form of characteristic baseline maps or profiles which have good diagnostic accuracy for the disease to be diagnosed (such as NFLT thinning for the diagnosis of glaucoma).

When using the individualized baseline model to calculate individualized baseline maps or profiles, the input features of the eye anatomy include maps or profiles that can be extracted from an OCT image or numerical values provided as input. Those eye anatomy features are also required to be insensitive to the disease of interest because they will be used to reconstruct the individualized baseline of OCT characteristics of the specific eye in a healthy pre-disease state. They are also required to be factors that affect the spatial distribution of OCT characteristic maps, in one approach.

In the case of glaucoma, the following eye anatomy features may be selected as model inputs which are insensitive to glaucomatous disease progression: vascular pattern map or profile, retinal elevation map or profile, optic disc shape map or size, axial length, spherical refractive error, and disc size. Among them, vascular pattern, retinal elevation, and disc shape are represented as maps or profiles. The vascular pattern can include the shape of large vessels, the retinal elevation can be captured by the Bruch's membrane elevation, and the disc shape can be represented as the masked shape of the Bruch's membrane opening. Each of these maps or profiles can be extracted from OCT scan data. The axial length and refractive error values can be estimated based on other eye anatomic features or can be measured and entered directly.

FIG. 1B depicts example eye anatomy maps which may be used as inputs to the model 100 of FIG. 1A, in accordance with various embodiments. These are eye anatomy maps which are not affected by a disease of interest such as glaucoma. The left-hand image depicts a large vessel mask. The right-hand image depicts a Bruch's membrane elevation map masked by the Bruch's membrane opening (or disc shape, shown as a black mask in the center).

FIG. 1C depicts a table of example inputs and outputs of the model 100, in accordance with various embodiments. In addition to anatomy-derived inputs and outputs, demographic information such as age, gender, race, and other confounding factors, can also be selected as conditions of the latent mapper. Furthermore, image quality, which can be quantified by signal strength index and which is known to be a confounding factor of NFLT and other OCT variables, may also be used as an input condition of the latent mapper.

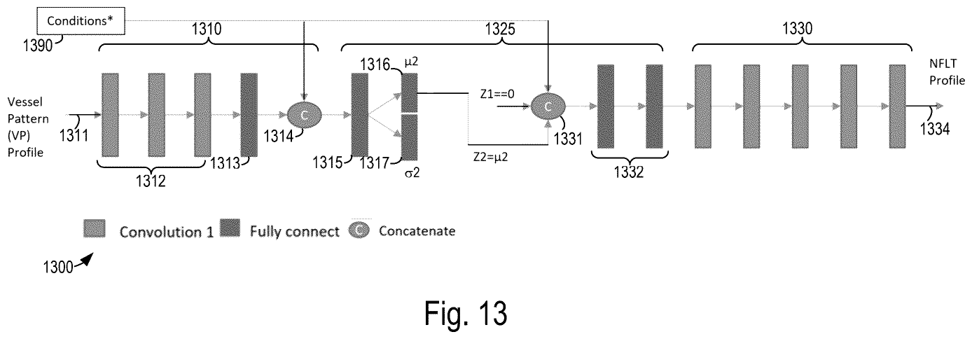

A CVAE model is represented as an example of the generalized individualized baseline model in FIG. 13, discussed further below.

However, it should be appreciated that other models and training processes can be used. For example, with reference to FIG. 13, the VP encoder can be trained with VAE to reconstruct the VP map, the NFLT decoder can be trained with another VAE for the NFLT map, and the latent mapper model can be a U-net model, trained in a full model of the pretrained VP encoder and NFLT decoder.

The outputs of the individualized baseline model generally apply to any of several OCT or OCT angiography maps or profiles that are useful for glaucoma diagnosis. For example, the individualized model can be used to generate a customized baseline reference for OCT measurements, like peripapillary NFL thickness, optic disc rim width, optic disc cup-to-disc ratio, macular ganglion cell complex thickness, ganglion cell-inner plexiform layer, or ganglion cell layer thickness. The individualized baseline model may also be used with nerve fiber layer reflectance variables, such as nerve fiber layer reflectance ratio in the peripapillary area or the macular area. Similarly, the individualized baseline model may also be configured to use OCT angiography (OCTA) maps as input or output, such as nerve fiber plexus capillary density (NFLP-CD), superficial vessel complex vessel density (SVC-VD), or ganglion cell complex plexus vessel density (GCLP-VD).

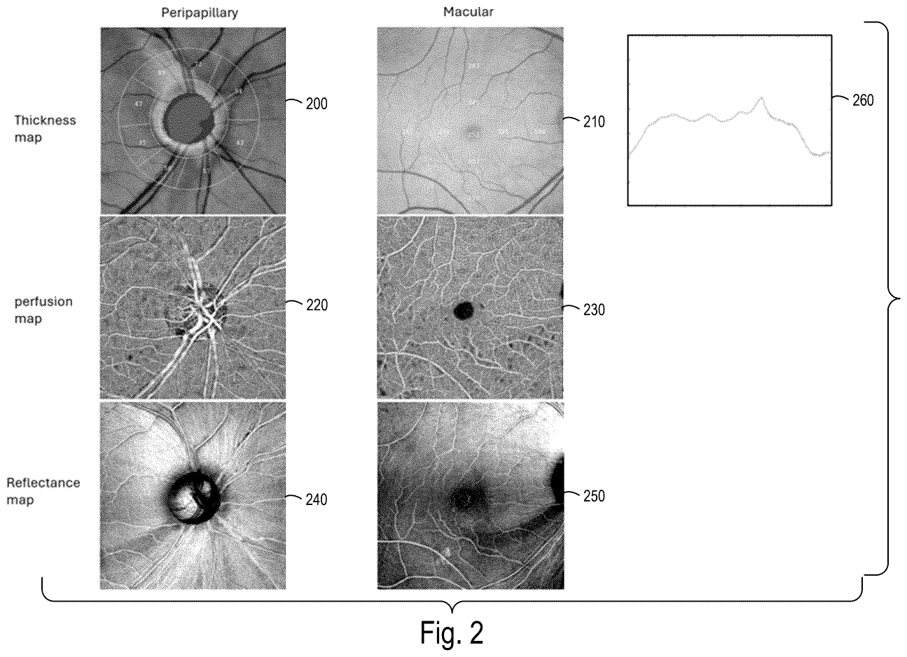

FIG. 2 depicts an example of different eye anatomy maps for use in the model 100 of FIG. 1A, in accordance with various embodiments. The examples include OCT and OCT angiography characteristic maps/profiles of a perimetric glaucoma eye. Notice there is apparent NFL bundle damage in the inferior hemisphere in all maps/profiles. In the predicted individualized baseline, it is expected that damage will not be presented because it is a prediction of a normal reference.

Image 200 depicts a peripapillary nerve fiber layer thickness map, image 210 depicts a macular ganglion cell complex thickness map, image 220 depicts a peripapillary nerve fiber layer plexus capillary density map, image 230 depicts a macular ganglion cell plexus vessel density map, image 240 depicts a peripapillary nerve fiber layer reflectance map, and image 250 depicts a macular nerve fiber layer reflectance map. Image 260 depicts a disc rim profile.

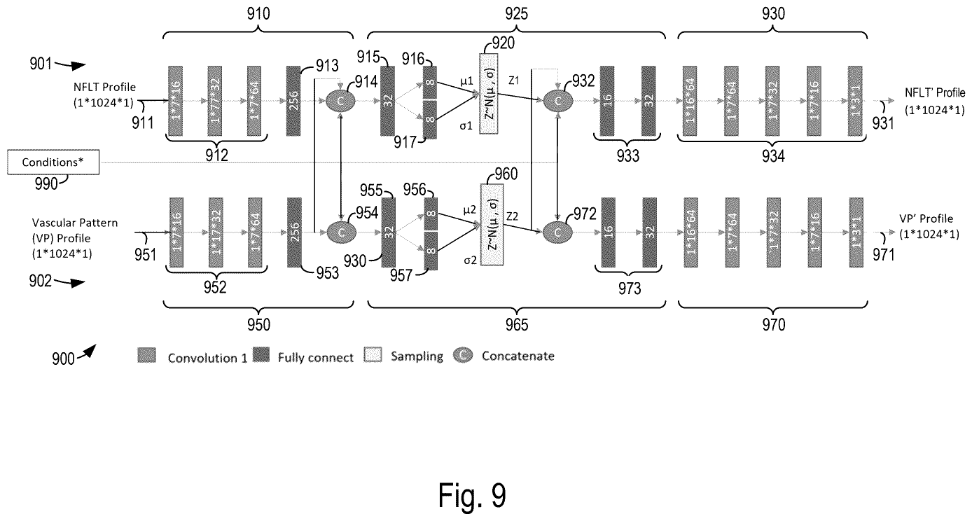

The generalized individualized baseline model can be trained with any dataset with OCT or OCT angiography of healthy controls. It can be either a relatively large dataset with enough healthy eyes to train the model from scratch or a smaller dataset to train the model using transfer learning. The requirement for the dataset size in transfer learning is different. In the case of the VP decoder of FIG. 9, for example, the VP decoder may require little modification and a small dataset to train. The size requirement of the dataset to train the OCT/OCTA map decoder depends on the similarity of the new parameter to the parameter in the pretrained model. However, the decoder may be trained on a dataset without labelling; therefore, it is easy to obtain in many situations. Only the latent mapper needs a reasonable size of dataset with clean, healthy labels to ensure the mapping of the input latent space into normal eyes for reconstruction.

In summary, the input of the generalized individualized baseline model can include eye anatomy features which are not sensitive to a disease of interest, such as demographic information and scan quality. The output of the generalized individualized baseline model is a diagnostic OCT or OCTA characteristic which are predicted to be the normal reference (or pre-disease baseline) to match the specific eye anatomy and conditions. To detect if an eye has a disease, the solution measures the difference between the predicted baseline OCT/OCTA map and the measured OCT/OCTA map.

### Example 1

The inventors have hypothesized that an individualized baseline NFLT profile would serve as a better reference for detecting pathological thinning associated with glaucoma and other optic neuropathies. As used herein, an individualized baseline is defined as a customized normal reference that accounts for individual eye characteristics such as the vascular pattern and the transverse optical magnification, which can be represented by its determinants: the axial length (AL) and spherical equivalent (SE) refractive error. The inventors have further hypothesized that it should be feasible to generate an individualized NFLT baseline using the map of major retinal blood vessels (which can be extracted from the same OCT scan used to produce the NFLT map) augmented with AL, SE, and demographic information.

In the following detailed example, three deep learning models are developed, based on how the AL/SE information was incorporated into the model: 1) no AL/SE data used; 2) measured AL/SE values provided as conditions; 3) AL/SE values estimated by a rCNN and provided as conditions. Their performance is compared to a regression model. The performances for these various baseline references are compared by the prediction error, the root-mean-square of the difference between the true NFLT parameters and the generated baselines. False positive rate (FPR) of detecting significant NFLT loss was also calculated, stratified by myopic refractive error, to assess the effectiveness of reducing the “red disease” problem. The difference between the reference NFLT with the 5 percentile cutoffs is used as a preliminary estimation of potential gain in glaucoma diagnostic sensitivity.

### Methods

Participants: Two different datasets were used in the present study, as follows:

Hong Kong Dataset: This cross-sectional study included participants from the Hong Kong FAMILY Cohort, a large territory-wide random sample of occupants from several Hong Kong districts. The Institutional Review Board of the University of Hong Kong approved the study, which adhered to the Declaration of Helsinki. Comprehensive details of the recruitment process and cohort characteristics were previously reported. In brief, all participants older than 18 were invited to participate and provided consent prior to enrollment. All participants received a comprehensive ophthalmic examination, including visual acuity, subjective refraction, perimetry, keratometry, pachymetry, axial eye length, intraocular pressure, slit-lamp examination, and indirect ophthalmoscopy. The axial eye length was measured with an ocular biometer (AL-Scan, Nidek, Gamagori, Japan).

Only healthy (e.g., glaucoma free) eyes were included in this study, and either one or two eyes were included per participant. The exclusion criteria were the following: subjects with a history of glaucoma, abnormal test in frequency doubling technology (FDT) perimetry or fundus examination (disc, macula, or vessels), elevated intraocular pressure (>21 mm Hg), enlarged cup-to-disc ratio (>0.7), pseudophakia, missing data.

Casey Eye Institute Dataset: This case-control study was performed at the Casey Eye Institute (CEI), Oregon Health & Science University. The research protocol was approved by the institutional review board at Oregon Health & Science University and adhered to the tenets of the Declaration of Helsinki. Written informed consent was obtained from each participant.

Participants were part of the “Functional and Structural Optical Coherence Tomography for Glaucoma” study. The inclusion criteria for normal control were (1) no history of glaucoma, retinal pathology, or current corticosteroid use; (2) no history of ocular hypertension as defined by IOP≥22 mmHg; (3) normal Humphery 24-2 VF test; (4) normal optic nerve head and NFL appearance on funduscopy; (5) symmetric optic nerve head appearance between both eyes; (6) central pachymetry >470 μm. The exclusion criteria were (1) best-corrected visual acuity less than 20/40; (2) previous intraocular surgery except for uncomplicated cataract extraction with posterior chamber intraocular lens implantation; (4) any diseases that may cause VF loss or optic disc abnormalities; (5) narrow anterior chamber angle by gonioscopy. Only one eye of each participant received OCT scanning and analysis. This CEI dataset was introduced to obtain independent validation of models trained on the Hong Kong dataset.

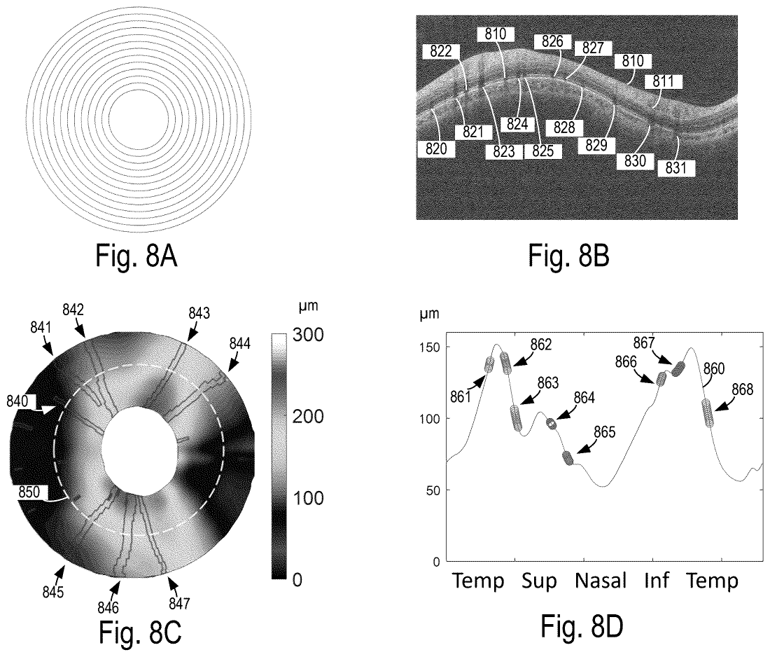

OCT Measurements: The NFLT and disc size were obtained from an optic nerve head (ONH) scan using a commercially available spectral-domain OCT device (Avanti with Angio Vue OCTA, Visionix/Optovue Inc, Fremont, California, USA). The ONH scan contains 13 rings covering a 4.9 mm peripapillary area around the optic disc (FIG. 8A). Raw OCT images, boundary segmentation, signal strength defined by the signal strength index (SSI, 0-100), disc size defined by disc area in mm, and the NFLT profile at diameter D=3.4 mm were exported for data analysis in this study. An automated quality check algorithm was applied to OCT images to remove scans with poor SSI (SSI<35), retina cropping, or extremely low NFLT (NFLT value lower than 4 times the population Standard deviation in healthy eyes). A previous study showed that SSI and disc size were also predictive factors of NFLT.

The vessel location was detected using the shadow of large vessels in the inner retina on the retinal pigment epithelium (RPE) (FIG. 8B). An en face map of vessel intensity in the RPE complex was reconstructed from the vessel shadow profile on the 13 rings of the ONH scan (FIG. 8A). A level-set method was then applied to detect large vessels (FIG. 8C).

The vascular pattern, NFLT, and boundary elevation profiles on different circles around the disc center were resampled from maps. Primarily, the NFLT profile and the vascular pattern profile at diameter-3.4 mm were used as input for the generative deep learning model (FIG. 13). The inner limiting membrane (ILM) and RPE elevation maps were also reconstructed, in a manner similar to the NFLT map.

Generative Deep Learning Models: A conditional variational convolutional autoencoder (CVAE) was used to encode and generate the NFLT profile. A second CVAE was used to encode and reconstruct the vascular pattern. (See, e.g., FIG. 9). The generation of NFLT by the first CVAE was conditioned on the encoded vascular pattern from the second CVAE. Both CVAEs were conditioned by factors associated with an individual person, eye, and scan: age, sex, AL, SE, disc size, and scan signal strength. The variational autoencoder (VAE) architecture was selected to address the issue of non-regularized latent space in the autoencoder and provide generative capability to the entire space. VAEs blend deep learning with probabilistic reasoning. Like an autoencoder, they have an encoder and decoder, but instead of just copying data, they learn the underlying probability distribution. This involves approximating the true distribution and minimizing the difference between them using a simpler one. CVAE is a special type of VAE that adds individual factors to control the NFLT generation. Advantageously, two CVAEs were connected by using the hidden space of the CVAE of the vascular pattern as a conditional input of the NFLT CVAE. The latent space of the vascular pattern CVAE provided vectors corresponding to an individual vascular pattern. Inclusion of those vectors made the generated NFLT profile match the specific vascular pattern.

Three AI-based models were designed, referred to as BASE, MAG and REG, depending on the use of magnification factors. The details are included in the next sections.

Models in the Training Stage: The BASE model described in this example included two parallel CVAEs (FIG. 9). Each CVAE has three parts: an encoder, a decoder, and a sampling block. In the encoder, a convolutional neural network (CNN) with several convolution blocks was used to convert profiles from one dimension in space into vectors in a higher dimension and reduce the transverse size to 1. A concatenate layer was then used to combine the output of CNN with conditions. A fully connected network converted the concatenated vector into vectors representing the μ (mean) and σ (standard deviation) in the latent space. The sampling blocks created random variables z~N(μ, σ). In the decoder, the random variables were again concatenated with conditions. Then, a fully connected network, followed by several transverse convolution blocks, was used to reconstruct the profile. Conditions were different between the two CVAEs. The vascular pattern CVAE used demographic information, such as age and gender, as a condition. The NFLT CVAE used demographic information, plus the output from the vascular pattern CVAE, as the conditions. In the NFLT encoder, the output of the CNN of the vascular pattern CVAE was used as a condition for the vascular pattern. In the NFLT decoder, the output of the sample layer of the vascular pattern CVAE was used as a condition for the vascular pattern.

The MAG model (FIG. 11) used in this example was structurally identical to the BASE model, but the conditions were updated to include magnification-related information, such as AL, SE, and disc area, SSI, in addition to the demographic information.

Because AL and SE may not be available in a commercial OCT system, the REG model was also developed to investigate the effect of including estimates of these parameters. In this example, the REG model used predicted values of AL and SE that were calculated based on OCT data (FIG. 12). The magnification information had been estimated from the vascular pattern using disc photography in the literature. A CNN for regression (rCNN) was used to predict the AL and SE using information from OCT and demographic information (FIG. 12). The rCNN includes convolution blocks and fully connected networks. The input of rCNN included the vascular pattern map (FIG. 8C) and the RPE elevation map. The ILM elevation map was not used as input in the REG model because glaucoma would change the characteristics of the ILM surface. Due to the limitation of the scan pattern, the available maps around the disc center (diameter=2~4 mm) were used. The rCNN was trained using the actual AL and SE data. Once trained, the AL and SE outputs of the rCNN were used to condition the CVAEs in the MAG model.

Loss Functions: The training of the above-described models is based on the minimization of a loss function. The loss function of a CVAE is a summation of reconstruction loss and regularization loss. The reconstruction loss included the mean square error (MSE, for continuous variables) or binary cross entropy (BCE, for binary variables) between the input and the estimated profile, while the regularization loss included Kullback-Leibler divergence (KL) between the distributions represented by the latent vector and a standard Gaussian distribution. The loss function of the rCNN is a summation of MSE of all outputs. Therefore, the loss function of the REG model was the summation of the loss functions of 2 CVAES and 1 rCNN.

$$Loss = {MSE}_{N F L T} + B C {E}_{V P} + K {L}_{V P} + {KL}_{NFL} + {MSE}_{A L} + M S {E}_{S E}$$

In this loss function equation, the subscripts are defined as follows: NFLT refers to the CVAE for the NFLT profile, VP refers to the CVAE for the vascular pattern profile, and AL and SE refers to the rCNN.

The loss function of the BASE and MAG models is similar to the above formula but removes the last two MSE terms corresponding to the loss function of the rCNN.

$$Loss = {MSE}_{NFLT} + B C {E}_{V P} + K {L}_{V P} + {KL}_{NFL}$$

Models in the Generating Stage: In the generating stage, only the vascular pattern CVAE encoder and the NFLT CVAE decoder were used (FIG. 13). To generate the baseline NFLT value of the individual eyes, the input of the NFL-CVAE decoder (Z1) was set to 0. If Z1 is sampled from N(0, 1), this allows the generalization of a group of random vectors. If they are combined with a set of conditions, the NFLT decoder would generate a group of NFL thickness profiles corresponding to the same set of conditions. The variation in NFL thickness profiles will not be due to glaucoma damage, as the module is only trained on healthy eyes. They can then be averaged to get a normal reference for this given condition set. However, the average can also be approximated by using μ1=0 and σ1=0, which only need to run the module once, therefore significantly reducing the calculation cost. So, Z1 is forced to =0 to generate the normal reference.

Similarly, the output μ2 vector from the vascular pattern encoder is used directly as Z2, indicating the vascular pattern part in the NFLT decoder's conditions. Note that μ2 was calculated, different from 0, and represented the specific vascular pattern. Using the above two simplifications, a normal reference matching the individual vascular pattern and other conditions is generated.

In the generating stage, the BASE model included demographic information and the u vector form vascular pattern encoder; the MAG model included all conditions in the BASE model plus AL, SE, and disc area; the REG model used similar conditions as the MAG model but replaced the AL and SE by the predicted value from the rCNN.

Normalization of Input: Glaucoma causes attenuation of retinal vascular caliber, which may affect the generation of individualized NFLT baseline reference and reduce the sensitivity of detecting disease. To avoid this potential pitfall, the vessel size of all eyes is normalized. This normalization approach had the effect of resizing each vessel proportionally and maintained a constant ratio of pixel numbers between vessels and non-vessels.

All inputs were normalized according to their mean and standard deviation to reduce the scale difference among features. The normalization accelerated and stabilized the learning process and avoided the problem of exploding gradients in the regression network.

When both the left and right eyes were used for the same participant, the learning weight of each eye was halved in the training stage to equalize each participant's weight.

Training Algorithm and Parameter: In the training, the Adam optimization algorithm (decay=0.001) was chosen to update network parameters. Dropout (0.5) and L2 (0.02) regularization were used to reduce overfitting. The initial learning rate was set to 0.001.

Multiple Linear Regression: The normative reference for NFLT can be improved by accounting for the effect of demographics, scan quality, and magnification. To assess the performance of this approach, multiple linear regression (MLR) with a mixed-effect model was used. Based on previous studies, age, sex, and SSI and AL were identified as the optimal combination of independent variables in the model. A broken stick (or segmented) regression model related NFLT to AL. With the breakpoints set at emmetropia (SE=0 diopters), the broken stick model divided healthy eyes into two segments: the hyperopia segment (SE>0) and the myopia segment (SE<0). The MLR was estimated for the overall average, sectoral averages, and each point on the NFL profiles. The normal reference is constructed by averaging all eyes after adjusting NFLT to a reference age/sex/SSI/AL based on the MLR. When the prediction error was computed or the abnormality of a testing eye was checked, the NFLT was adjusted using the same scheme.

Statistical Analysis: In total, five models of normal reference were compared in this study:
1) population average without any adjustment (Average); 2) adjusted based on the multiple linear regression (MLR); 3) generated by the deep learning model without magnification information (BASE); 4) generated by deep learning model with magnification information, using true AL and SE directly (MAG); and 5) generated by the deep learning model with AL and SE predictand by rCNN, (REG).

Based on the Hong Kong dataset, five-fold cross-validation was used to test the performance of the fully trained models. For each fold, 80% of the eyes were used to train the deep learning/MLR models and the remaining 20% of the eyes to test the performance of the trained model. The final performance was pooled from all folds. In the training of deep learning models, 10% of the training set was reserved for internal validation (not to be confused with the five-fold validation) to avoid overfitting.

The prediction error was estimated as the root-mean-square of the difference between the true value and the predicted value using a mixed-effect model. The false positive rates (FPR) were compared among models, using a generalized linear mixed-effect model (GLMM) equivalent to the McNemar test. All analyses were done in MATLAB R2021b with the Statistics Toolbox and deep learning toolbox. Mixed-effect models were used to address between-eye correlation when applicable.

The models trained on the Hong Kong dataset were also applied to the CEI dataset. The prediction errors between the predicted normal reference and the tested eyes were estimated for the overall, quadrant, and profiles of NFL thickness. To compensate for the difference in NFL thickness due to race in two datasets, mainly East Asians in the Hong Kong dataset and multiple races in the CEI dataset, the individualized baseline was proportionally adjusted according to the ratio of the population average of NFL thickness profile between the emmetropia eyes in the two datasets. To compare the prediction error among models, a mixed-effect model was fitted to each data set, followed by Dunnett's (post-hoc) test.

### Results

### Characteristics of the Study Participants:

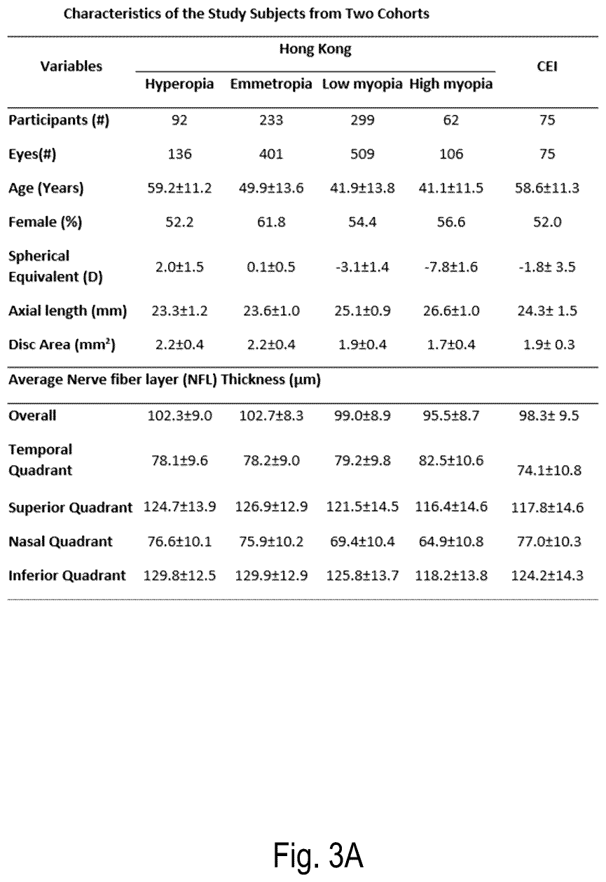

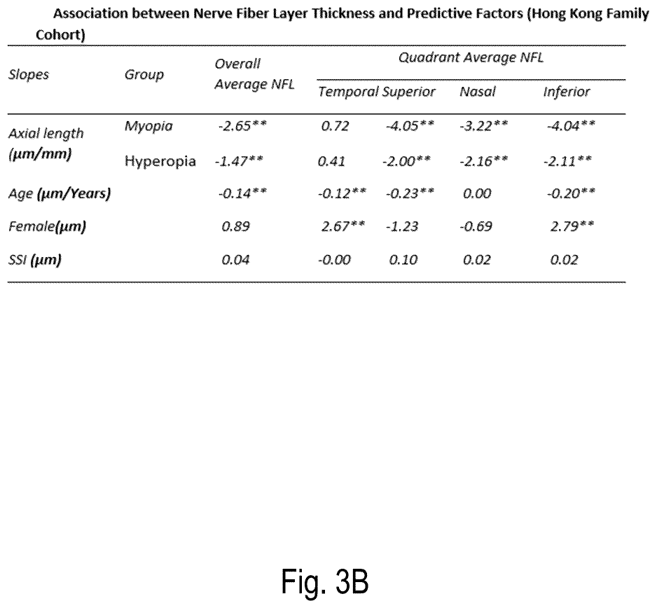

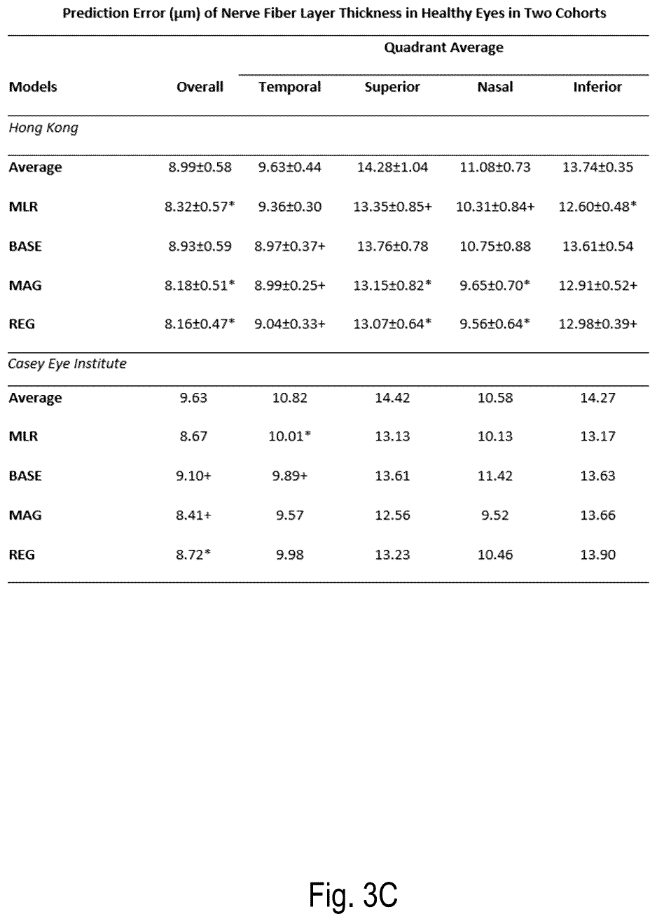

FIG. 3A depicts a table of characteristics of study subjects from two cohorts, in accordance with various embodiments. The cohorts are the Hong Kong FAMILY and CEI. The variables include number of participants, number of eyes, age, percent female, spherical equivalent, axial length and disc area. The eyes are classified into subgroups of hyperopia, emmetropia, low myopia and high myopia, for the Hong Kong cohort. The average NFLT is determined for the temporal, superior, nasal and inferior quadrants, and an overall MFLT is determined as an average of the four quadrants. In FIGS. 3A, 3C, 6B and 7, values for continuous variables are depicted as mean±standard deviation.

A total of 1152 healthy eyes from 686 participants with valid age, gender, AL, SE, and NFLT profiles were selected from the Hong Kong dataset. Eyes were divided into four subgroups: 106 high myopia (SE<-−6 D), 509 low myopia (−6 to −1 D), 401 emmetropia (−1 D to 1 D), and 136 hyperopia (>1 D). As expected, myopic eyes had longer AL, smaller disc size, and thinner NFL. Myopic eyes were also younger.

A total of 75 normal eyes from 75 participants were selected from the CEI dataset (last column, FIG. 3A). The CEI dataset is relatively older and has fewer female participants than the Hong Kong dataset. A wide range of myopia eyes were included in the CEI dataset (SE=−14.5~5 D, AL=21.7~29.0 mm), but the average SE, AL, and Disc size of the CEI dataset were between emmetropia and low myopia in the Hong Kong dataset.

### Training of Regression and Deep Learning Models:

FIG. 3B depicts a table of associations between nerve fiber layer thickness (NFLT) and predictive factors from the Hong Kong FAMILY cohort of FIG. 3A, in accordance with various embodiments. The table depicts slopes including axial length for low myopia and hyperopia subgroups, age, percent female and signal strength index (SSI). The average NFL is determined for each of the four quadrants. In the table, *p<0.01, **p<0.005.

The SSI of an OCT scan is a quantitative measure of image quality, ranging from 0 to 100 on Optovue® systems or 0 to 10 on other systems such as Cirrus®. It reflects the intensity of reflected light, with higher scores indicating better quality.

Accounting for multiple comparisons of four quadrant values plus the overall value, the Bonferroni correction was used to set the p-value cutoff at 0.01 for statistical significance. Gender difference is calculated by female-male. +Slopes against axial length were different between myopia segment and hyperopia segment using the broken stick model, which divided the healthy eyes into two segments with break point at the spherical equivalent refractive error=0.

Based on the Hong Kong dataset, the association of NFLT with predictive factors varied between quadrants. In the MLR with a broken stick model, NFLT was significantly associated with AL (p<0.001) for overall average and superior, nasal, and inferior quadrants. Significant associations were also found for age and gender, with different quadrant distributions. Those slopes based on the MLR were used to adjust the NFLT in later analyses.

In the REG model, the correlation coefficient between the predicted value and the ground truth is 0.59±0.05 (p<0.001) for AL and 0.54±0.05 (p<0.001) for SE, based on 5-fold cross-validation. The correlation is moderate and much smaller than the correlation in the training, which was usually above 0.70. This indicated overfitting in the regression model. Experiments by choosing a larger dropout rate, larger L2, or shallower network reduced the overfitting in the training, but the correlation in the test dataset was in the same range.

The estimation of binary cross-entropy loss of the vascular pattern of three deep learning models was similar (0.372~0.375). The MAG and REG models had smaller root mean square errors of NFL thickness prediction (8.27 and 8.20 μm) than the BASE model (8.95 μm).

### Prediction Error Between Actual NFLT and the Predicted Baseline Reference:

FIG. 3C depicts a table of prediction errors in μm of NFLT in healthy eyes in the two cohorts of FIG. 3A, in accordance with various embodiments. The models include MLR, BASE, MAG and REG and an average result of the four models. Additionally, *, p<0.0125 and +, p<0.05, compared to the Average model.

A smaller NFLT prediction error in healthy eyes means that a tighter diagnostic threshold can be used at a given specificity level. This helps to detect glaucoma at an earlier stage when NFLT thinning is more subtle. The difference between the actual individual NFLT values and the predicted baseline reference for each of the 5 models were calculated. The prediction error of the more advanced models was then compared to the simplest model (unadjusted population average), for which the prediction error was simply the population standard deviation in the test dataset.

Based on the Hong Kong dataset, the MAG and REG models had the smallest prediction error for the overall and quadrants. They were significantly lower than the normal reference based on the population average. However, they were only significantly better than the MLR model in nasal (p<=0.01), but borderline for others (p<0.10). The BASE model did not significantly reduce the prediction error compared to the population average without adjustment.

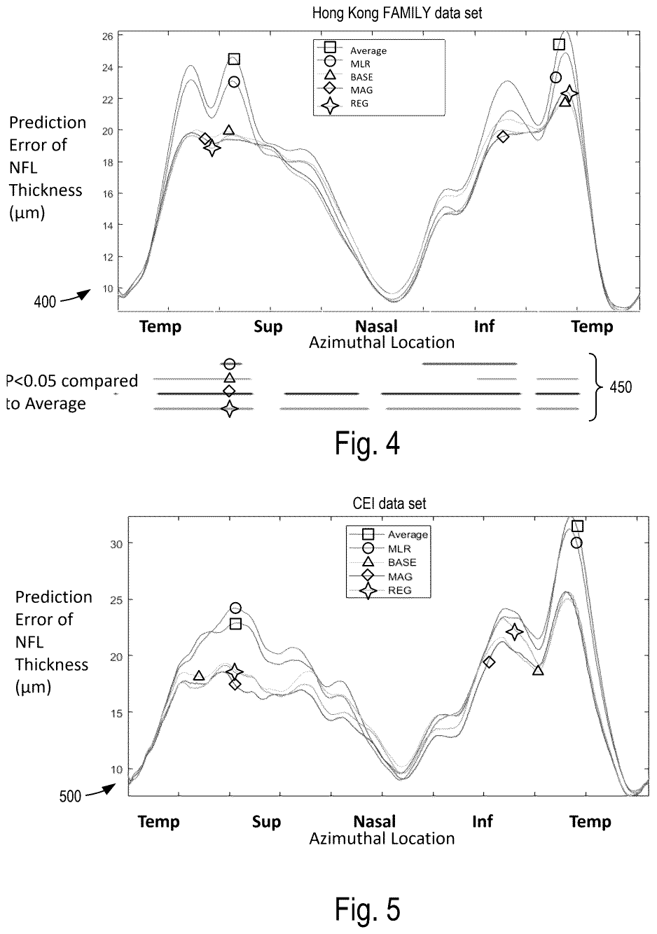

FIG. 4 depicts prediction error profiles (plots 400) and significance masks (plots 450) for p<0.05 compared to a population average over five models, for the Hong Kong FAMILY cohort of FIG. 3A, in accordance with various embodiments.

The prediction error is based on the root-mean-square of the profile difference (true NFL profiles-predicted normal reference or individualized baseline) for five models: population average: no adjustment; MLR; normal reference adjusted based on multiple linear regression; BASE model: deep learning models using conditional variance autoencoder; MAG: BASE model plus magnification information; REG: BASE model with magnification estimated with a regression convolutional neural network (rCNN); p-Value<0.05 were used to check if there was a significant difference in prediction errors between the average and other models.

Based on the Hong Kong Dataset, the original (population average) model had large prediction errors near the prominent NFLT peaks. This was expected as the location of these peaks (arcuate bundles) can vary between individuals. Compared to the original and MLR models, both the MAG and REG models greatly reduced the prediction error of the NFLT profile near the arcuate bundles. The BASE model also reduced the prediction error to a small degree. The MLR model did not significantly reduce the prediction error of NFLT at the arcuate bundles. This was expected as the inputs to the MLR model did not contain information on the location of the arcuate bundles.

The thickness peaks in the NFLT profile are at the superior and inferior arcuate bundles that generally collocate with the major arcade vessels. Thus the peripapillary vascular pattern measured on the OCT can be used to predict the NFLT peak location. The vascular pattern also contains information on the papillomacular axis, which also influences the NFLT pattern. Disc size is another measurable individual characteristic that influences NFLT.

FIG. 5 depicts present prediction error profiles (plots 500) over five models, for the Casey Eye Institute (CEI) cohort of FIG. 3A, in accordance with various embodiments. Similar trends were observed based on the CEI dataset. All models showed lower prediction error for overall and quadrants than the population average. The MAG models had the smallest prediction error among other models, except for at the inferior quadrant. For the Profile, the MAG deep learning model showed the smallest prediction errors in the superior quadrant and inferior-temporal sectors. All three deep learning models showed significantly smaller prediction error compared to both MLR and average models (root mean squares=16.7 μm, 16.0 μm, and 16.6 μm vs. 18.7 μm and 18.8 μm, p<0.001). The prediction errors from the CEI dataset were larger than those from the Hong Kong dataset for all parameters and all models, including the population average. This indicates a greater variability in the CEI dataset, possibly because it is multiracial. The effect of refractive error on the rate of false-positive abnormality in the CEI dataset was not analyzed because of the lack of highly myopic eyes.

### False Positive Rate:

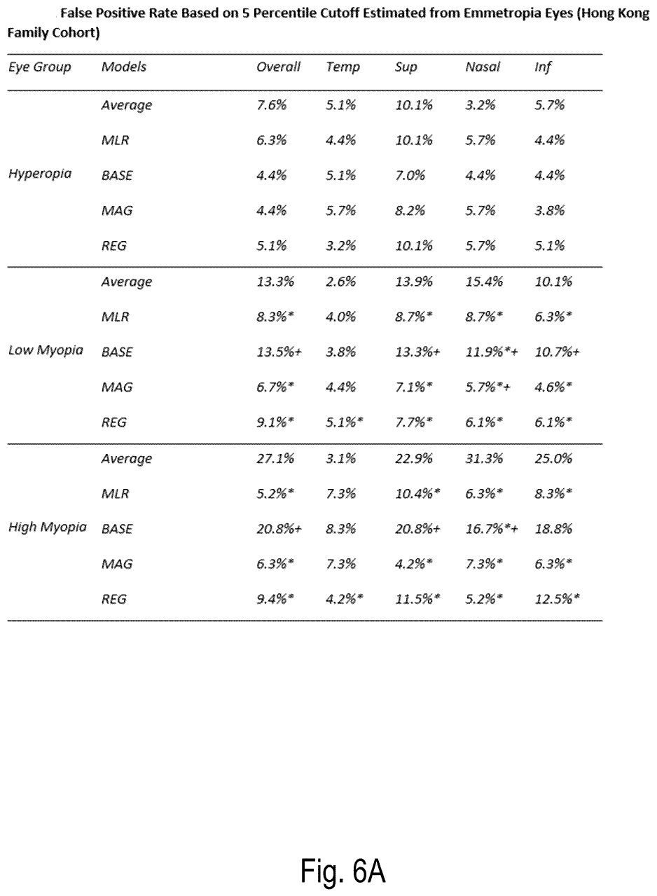

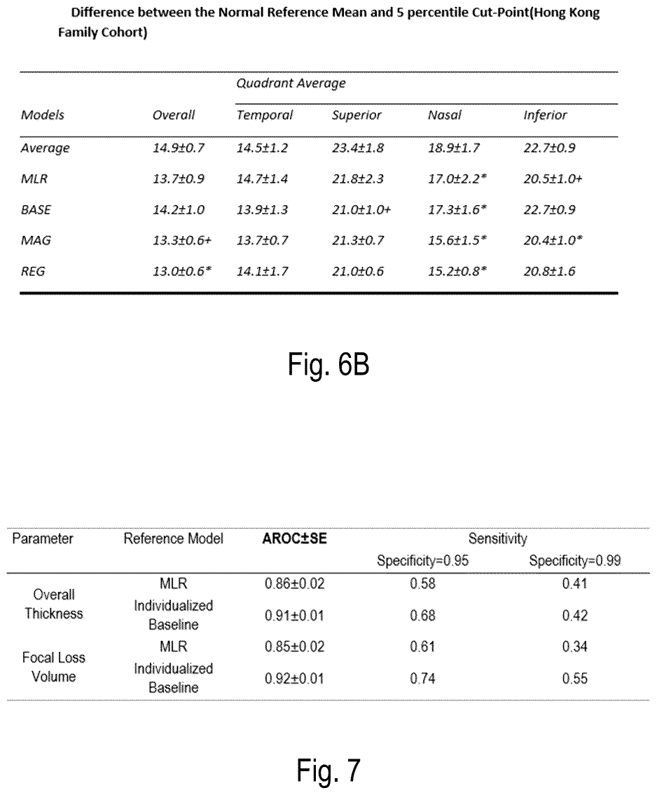

FIG. 6A depicts a table of false positive rate based on a 5-percentile cutoff estimated from emmetropia eyes for the Hong Kong FAMILY cohort of FIG. 3A, in accordance with various embodiments. Additionally, *p-value<0.0125 comparing to average, +p-value<0.0125 comparing to the MLR method.

False positive rates (FPR) were based on eyes with thickness below the 5th percentile cutoff of the normal reference. The cutoff was estimated from the histogram of the original NFLT or the adjusted NFLT in the emmetropia group. The REG model significantly reduced the FPR in the myopia and high myopia groups compared to the original value, except in the temporal area. The REG model had a similar performance on FPR compared to MLR or MAG model in most of the parameters (p>0.05). However, the MLR and MAG models showed more consistent FPR in all groups. In hyperopia, all models had similar FPR (p>0.05). For the temporal quadrant average of NFLT, the original value showed FPR significantly less than 5% in myopia groups, which might be due to the temporal NFL being less affected by magnification and the population variance in myopia groups being significantly lower than the emmetropia group.

### Difference Between Normal Reference and Five Percentile Cutoff:

FIG. 6B depicts a table of differences between a normal reference mean and a 5 percentile cut point for the Hong Kong FAMILY cohort of FIG. 3A, in accordance with various embodiments. The models include Average, MLR, BASE, MAG and REG. A value is given for each of the four quadrants, with an overall or average value. Additionally, *p<0.0125, +p<0.05 compared to the population average model.

Due to the lack of verified glaucoma eyes in the Hong Kong dataset, the diagnostic sensitivity cannot be evaluated directly. Instead, the difference between each model's normal reference/baseline and the 5-percentile cut point was estimated. A tighter (smaller) difference indicated that glaucomatous eyes at earlier stages with smaller loss of NFLT could be detected. For a population of glaucomatous eyes that includes those with early disease, the ability to detect smaller deviations from the healthy baseline would lead to better diagnostic sensitivity. MLR and all deep learning models reduced the difference between the median and the 5-percentile cutoff, compared to the population average. Among four adjusting models, two deep learning models with magnification information had smaller differences than other models, and the BASE model had the worst performance, though those comparisons were not statistically significant (p>0.0125).

FIG. 7 depicts a table of results from testing a nerve fiber layer thickness individualized baseline model with an Advanced Image for Glaucoma (AIG) study dataset, in accordance with various embodiments. To evaluate its ability to diagnose glaucoma, the nerve fiber layer thickness individualized baseline model was tested with the dataset of the Advanced Image for Glaucoma (AIG) study, which have 238 perimetric glaucoma (PG) and 210 healthy control (HC) eyes. The generated AI model was revised by adding a linear regression model to account for racial variance between the original training set to the test dataset. Comparing with a normal reference adjusted by multiple linear regression (MLR) model to compensate for age, axial length, and race, it was found that the individualized baseline significantly (p<0.05) improved the diagnostic accuracy for both overall average and focal loss volume, which measured the focal NFL loss.

The table confirms that an individualized baseline improves the diagnostic accuracy of focal loss analysis. Here, SE denotes standard error, AROC denotes area under receiver characteristic curve, PPG denotes pre-perimetric glaucoma, and PG denotes perimetric glaucoma. This demonstrates the effectiveness of the techniques on yet another dataset.

FIG. 8A-8D depict the nerve fiber layer (NFL) thickness map and vascular pattern. FIG. 8A presents a circular pattern of 13 rings covering a peripapillary area around the optic disc as utilized in ONH scans, in accordance with various embodiments. The ONH scan consists of circular scans (D=1.3~4.9 mm) covering the peripapillary area.

FIG. 8B presents a representative OCT B-scan with NFLT boundaries (solid lines 810 and 811) and vessel shadow detected (dots 820-831), in accordance with various embodiments. An OCT B-scan is a non-invasive, high-resolution cross-sectional image of tissue, used in ophthalmology to visualize layers of the retina, optic nerve, or cornea. It is created by combining multiple axial measurements (A-scans) along a linear path to produce a 2D image.

FIG. 8C presents a representative NFLT map and main vessel location map obtained from vessel shadow (example solid radial lines 840-847), where the center area masked the optic disc, in accordance with various embodiments. The shading represents the thickness and varies in a scale from 0-300 μm.

FIG. 8D presents a representative NFLT (RNFLT) profile (plot 860) and vascular pattern (sets of circles 861-868) that were resampled on the re-centered 3.4 mm circle (circular dashed line 850 in FIG. 8C), in accordance with various embodiments. The vascular pattern was used as a binary mask. The vertical axis depicts the thickness on a scale of 0-150 μm, and the horizontal scale depicts the quadrant, ranging from temporal (Temp) to superior (Sup) to nasal to inferior (Inf) and back to temporal.

FIG. 9 represents an overall structure of an example BASE model 900 in the training stage, consistent with FIG. 1A, in accordance with various embodiments.

In this example implementation, the encoder is implemented using CNN layers to convert the vascular pattern profile into features, and the features are then concatenated with numerical conditions. The latent mapper is an autoencoder that converts vascular pattern features and other conditions into NFLT features for the specific eye. The decoder is implemented by CNN layers to reconstruct the NFLT profile from the features. Moreover, the encoder, mapper, and decoder can be trained using a dual conditional variation autoencoder (see FIG. 9).

The training of the CVAE model was represented as an example of training the generalized individualized baseline model. In this example, notice the use of both encoders of NFLT and VP, and both decoders for the NFLT and VP in the training to benefit from the unsupervised training process in VAE by matching the input and output. In the latent mapper, the data flow is from the VP encoder to the NFLT decoder only, not vice versa. Using this feature, one can later drop the NFLT encoder by setting the output of this block to 0 and dropping models related to the VP decoder in the generating or test stage (FIG. 13).

The model includes a first CVAE 901 used to encode and generate the NFLT profile, and a second CVAE 902 used to encode and reconstruct the vascular pattern. The first CVAE includes an NFLT encoder 910 with an input 911, followed by a first latent mapper 925, followed by an NFLT decoder 930 having an output 931. The encoder 910 includes a set of convolution blocks in a CNN 912, followed by a network connect block 913, followed by a concatenate block 914. The first latent mapper 925 includes a network connect block 915, followed by network connect blocks 916 and 917, followed by a first sampler 920, followed by a concatenate block 932, followed by a set of network connect blocks 933. The NFLT decoder 930 includes a set of convolution blocks in a CNN 934.

Outputs μ1 and σ1 of the network connect blocks 916 and 917, respectively, are provided to the first sampler 920, which in turn provides an output Z1 to the concatenate block 932. The numbers in the blocks represent a data vector size, in an example implementation.

Similarly, the second CVAE includes a vascular pattern encoder 950 with an input 951, followed by a second latent mapper 965, followed by a vascular pattern decoder 970 having an output 971. The encoder 950 includes a set of convolution blocks in a CNN 952, followed by a network connect block 953, followed by a concatenate block 954. The second latent mapper 965 includes a network connect block 955, followed by network connect blocks 956 and 957, followed by a second sampler 960, followed by a concatenate block 972, followed by a set of network connect blocks 973.

Outputs μ2 and σ2 of the network connect blocks 956 and 957, respectively, are provided to the second sampler, which in turn provides an output Z2 to the concatenate block 972.

A set of one or more conditions 990 can be input to the concatenate blocks 914, 932, 954 and 972.

As mentioned, FIG. 9 represents the BASE model, which is a deep learning model using only demographic information. It has two parallel CVAEs, where each CVAE had three parts: encoder, latent mapper and decoder. In the encoder, firstly, a CNN was used with several convolution blocks to convert profiles from 1 dimension in space into vectors in a higher dimension and reduce the transverse size to 1. Then, a concatenate layer is used that combined the output of convolutional neural network with conditions. A fully connected network converted the concatenated vector into vectors representing the u and σ in the latent space. The sampling blocks created random variables z~N(μ, σ). In the decoder, the random variables were concatenated with conditions again. Then, a fully connected network, followed by several transverse convolution blocks, reconstructed the profile.

Conditions were different between the 2 CVAEs. The vascular pattern CVAE used demographic information, such as age and gender, as a condition. The NFLT CVAE used demographic information, plus the output from the vascular pattern CVAE, as the conditions. In the NFLT encoder, the output of the convolutional neural network of the vascular pattern CVAE was used as a condition for the vascular pattern. In the NFLT decoder, the output of the sample layer of the vascular pattern CVAE was used as a condition for the vascular pattern.

The input of the first CVAE 901 is a measured NFLT profile (NFLT) and the output is an estimated NFLT profile (NFLT′). The input of the second CVAE 902 is a measured vascular pattern profile (VP) and the output is an estimated vascular pattern profile (VP′).

The NFLT profile was used as a numeric array, and the vascular pattern profile was used as a binary array. The CVAEs are used to reconstruct both vascular pattern profile (VP) and nerve fiber layer thickness profile (NFLT). Conditions blocks were different for three deep models: BASE, MAG and REG. Note the VP encoder's output was also used as conditions of the NFLT encoder and decoders.

In sum, FIG. 9 provides an example of an individualized model for the RNFL, showing the NFLT thickness profile at a specified distance, e.g., D=3.4 mm, around the disc. In this example, the encoder includes the CNN layers to convert the VP profile into features and concatenates the features with numerical conditions. The latent mapper is actually the autoencoder part, with the random sampling seeds set to 0. The decoder is the CNN layers to reconstruct the NFLT profile. The encoder, latent mapper, and decoder can be trained as a single CVAE network or trained separately. One may also use other models; for example, the latent mapper model can be a U-net model.

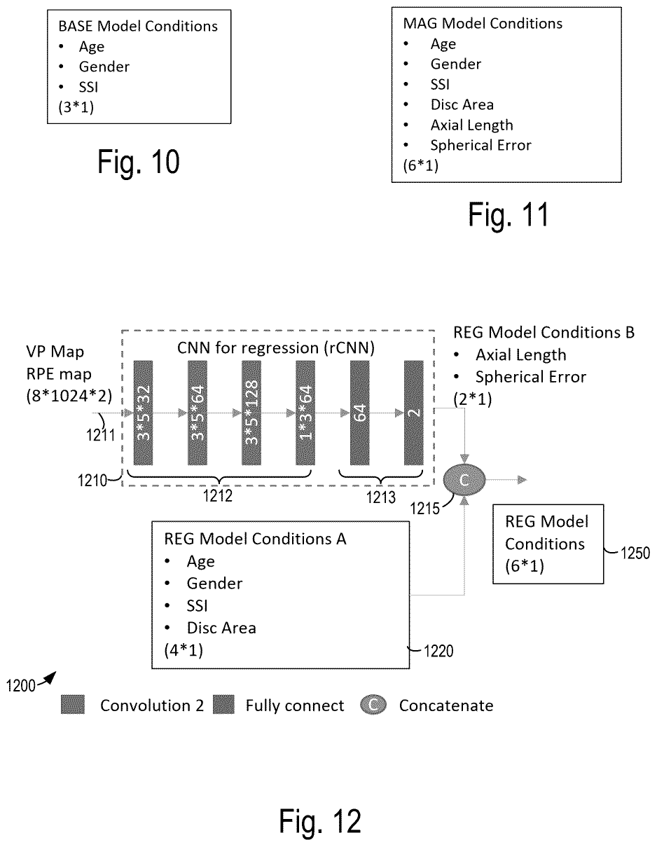

FIG. 10 represents example BASE model conditions including age, gender and signal strength index (SSI) conditions, in an example implementation of the conditions 990 of FIG. 9, in accordance with various embodiments.

FIG. 11 represents example MAG model conditions, including the conditions listed in the BASE model of FIG. 10, plus disc area, measured axial length (AL) and spherical equivalent error (SE), in another example implementation of the conditions 990 of FIG. 9, in accordance with various embodiments.

FIG. 12 depicts an example REG model 1200 used to provide REG model conditions 1250, in another example implementation of the conditions 990 of FIG. 9, in accordance with various embodiments. The conditions of the REG model are similar to the MAG model. However, AL and SE were predicted by an rCNN 1210. The rCNN used an RPE elevation map and vascular pattern map, such as based on a binary mask of vessels as shown in FIG. 8C. The rCNN 1210 includes an input 1211, followed by a set of convolution blocks in a CNN 1212, followed by a set of network connect blocks 1213, followed by a concatenate block 1215. The concatenate block 1215 receives model conditions from the rCNN, such as axial length and spherical equivalent refractive error, and other model conditions 1220 such as age, gender, SSI and disc area, and outputs the REG model conditions 1250.

FIG. 13 represents the overall structure of another example deep learning model 1300 in the generating stage, consistent with FIG. 1A, in accordance with various embodiments. The generating stage refers to using the model to evaluate data of an individual patient, after the model has been trained on a population of patients.

Generating the individualized baseline using deep learning models is much simpler compared to the training stage. Only the vascular pattern (VP) encoder and nerve fiber layer thickness (NFLT) decoder were used. The sampling layer is also removed in order to get the average NFL thickness, giving individual conditions. Therefore the input of the NFLT decoder is the combination of Z1=0, Z2=μ2 and other conditions.

This model includes a single CVAE having an encoder, latent mapper and decoder. The input of the CVAE is a measured VP profile and the output is a baseline NFLT profile of an individual patient for use in evaluating the patient.

The CVAE includes a vascular pattern encoder 1310 with an input 1311, followed by a latent mapper 1325, followed by an NFLT decoder 1330 having an output 1334. The encoder includes a set of convolution blocks in a CNN 1312, followed by a network connect block 1313, followed by a concatenate block 1314. The latent mapper includes by a network connect block 1315, followed by network connect blocks 1316 and 1317, followed by a concatenate block 1331, followed by a set of network connect blocks 1332. The decoder 1330 includes a set of convolution blocks in a CNN,

An output μ2 of the network connect block 1316 is provided to the concatenate block 1331.

A set of one or more conditions 1390 can be input to the concatenate blocks 1314 and 1331.

The output μ2 vector was used directly as Z2, indicating the vascular pattern part in the NFLT decoder's conditions. Note that u was calculated, different from 0, and represented the specific vascular pattern. Using the earlier two simplifications, a normal reference is generated matching the individual vascular pattern and other conditions.

### Discussion

In a proof-of-concept study, deep learning models were proposed to estimate an individualized baseline NFLT profile. By taking into account individual variations in the transverse optical magnification (related to axial eye length and refractive error) and anatomy (related to retinal vascular pattern), it was hypothesized that the individualized baseline would help distinguish real pathology from normal inter-individual variation and serve as a more reliable diagnostic reference than the simple population-average NFLT profile and sector averages. The deep learning models are based on CVAE, which estimates the individualized baseline NFLT profile using the vascular pattern profile derived from the OCT scan, as well as demographic factors (age, gender).

In the classification of artificial intelligence, CVAE is considered a type of probabilistic generative deep learning model, a category that also includes generative adversarial networks, diffusion models, and language models. The way that CVAE is used herein, however, is not probabilistic because the conditional input to the variational autoencoder is not random, but is determined by patient characteristics such as vascular pattern, RPE elevation map, axial length, and demographics. Thus, the individualized baseline NFL profile predicted by the model described herein is determined by the characteristics of the eye. Since the goal is to use the model to predict the individualized baseline NFL profile that would have existed without disease damage, it is advantageous that the model input is not affected by glaucoma or other diseases. This is an issue with the vascular pattern because glaucoma can attenuate retinal blood vessels. Thus, a step was inserted in the vascular pattern map generation to normalize the number of vessel pixels and prevent disease from affecting the overall vessel caliber.

Three deep learning models were developed based on CVAE. The BASE model was only based on vascular patterns and demographics. The other two generative deep learning models also incorporated information on the transverse optical magnification based either on actual measurements of AL and SE (MAG), or an rCNN that estimated AL and SE using OCT-derived vascular pattern map and RPE elevation map (REG). It was observed that the vascular pattern helped to align the NFLT peaks of the individualized baseline to test eyes. It was also found that individualized baseline models that incorporated actual or estimated magnification information significantly outperformed the population average in terms of the prediction error and reduced the false positive glaucoma diagnosis rate in the myopic eyes. The BASE CVAE did not perform as well as MAG and REG, demonstrating that accounting for magnification-associated NFLT variation was still essential to the performance improvement achieved by the disclosed deep learning approach.

Given similar performance, the REG model may be preferable to MAG because it does not require additional AL and SE measurements. The two models were found to have similar prediction errors, but MAG was more effective in reducing the false-positive diagnosis rate in myopic eyes.

The prediction error of the MAG model was smaller than that of the REG model in the validation with the CEI dataset. So, the REG model may need further improvement in generalizability. Such improvement may be possible by using wider maps of vascular pattern and RPE, as other investigators have found that wider-field disc photographs provided more accurate estimates of AL.

Besides CVAE, other methods can also account for individual variation and reduce prediction error. MLR was effective in reducing prediction error for overall and some quadrant NFLT, but not the NFLT profile. The ability of CVAE to reduce prediction error for NFLT profile in regions most susceptible to glaucoma damage (superior and inferior arcuate nerve fiber bundles) may be useful in improving the detection of focal glaucoma damage. The generative AI model may be better in predicting the NFLT profile because the location and bifurcation pattern of the NFL bundles may be correlated with the vascular pattern that serves as input to the CVAE. Other investigators have also used the retinal vascular pattern as input to machine learning algorithms to improve NFLT prediction. A potential advantage of the CVAE approach described here is that it provides highly individualized NFLT profile prediction. However, further studies are needed to compare the performance of the various approaches.

The results presented here showed that magnification-related information (AL, SE) and vascular pattern each significantly improved the accuracy of the individualized baseline reference generation. Either AL or SE alone also produced significant improvement (results not shown), but the combination was synergistic, and both clinical parameters are readily available. Therefore, results were presented for models using both AL and SE. While the individual evaluation of the importance of the other model inputs-age, sex, race, disc size, and signal strength-were not presented here, it was found that each of them improved the performance of the models to small degrees. The effect of these predictive factors has already been shown in previously published studies.

There are several limitations to this study. First, most eyes in the Hong Kong dataset were East Asian as the dataset was obtained from Hong Kong. Literature showed that East Asians had significantly or marginally thicker NFL than whites and blacks. A simple proportional adjustment for race was tried as a post-processing remedy, which works with reduced performance. It is possible that the local adjustment is different from the overall adjustment. Therefore, a better model might be achieved based on multi-racial training data and using race as a condition. The second limitation is that the model was trained with a single OCT system—the Avanti. Therefore, retraining would be needed to apply this approach to other OCT systems. The third limitation of this study is that diagnostic sensitivity has not been tested on a group of glaucoma patients. As a more specific normative reference, the individualized baseline will likely improve the accuracy of NFL focal loss analysis. The next step will be to develop an algorithm to detect focal NFL loss and assess its performance in glaucoma diagnosis. A fourth limitation is that deviation from normal reference does not necessarily indicate the presence of glaucoma—it could be due to other ocular diseases. Therefore, the individualized baseline described in this study would also need to be assessed in patients with other ocular comorbidities.

A potential pitfall of the disclosed approach is that eye diseases could affect the retinal vascular pattern and thereby the generation of the individualized NFLT baseline reference. Both glaucoma and diabetic retinopathy cause attenuation of retinal vascular caliber, which may cause the AI model to generate attenuated NFLT and reduce our ability to detect NFLT loss. To make the above-described model more disease-invariant, the vessel size was normalized to keep a fixed ratio of vascular to nonvascular pixels in the vascular pattern profiles. The effectiveness of this approach will be tested in future studies on glaucoma diagnostic accuracy.

A similar pitfall is that glaucoma could affect the ILM elevation map, leading to errors in the estimation of magnification-related factors AL and SE. For that reason, the RPE elevation map was used, which would not be affected by glaucomatous thinning of NFLT, to estimate AL and SE in the MAG model.

The above study has demonstrated that the generative deep learning approach can generate individualized NFLT profiles, sectors, and overall values that reduce prediction error relative to simple population averages. This approach can be extended to other OCT metrics and OCT angiography metrics, such as macular ganglion cell complex thickness, NFL plexus capillary density, or cup-disc ratio.

A generative deep learning AI model has been developed that can provide an individualized NFLT baseline using vascular patterns from OCT only. Compared to models based on multiple regression, the individualized baseline performed equally in reducing the population variance of global NFLT in healthy eyes or false positive rate in detected NFLT abnormality in myopia, but performed better in reducing the variance locally. The approach to constructing the individualized baseline could be extended to other OCT and OCT angiography metrics or characteristics such as vessel density (area/perfusion density), foveal avascular zone area/perimeter, and vessel complexity (fractal dimension).

### Example 2

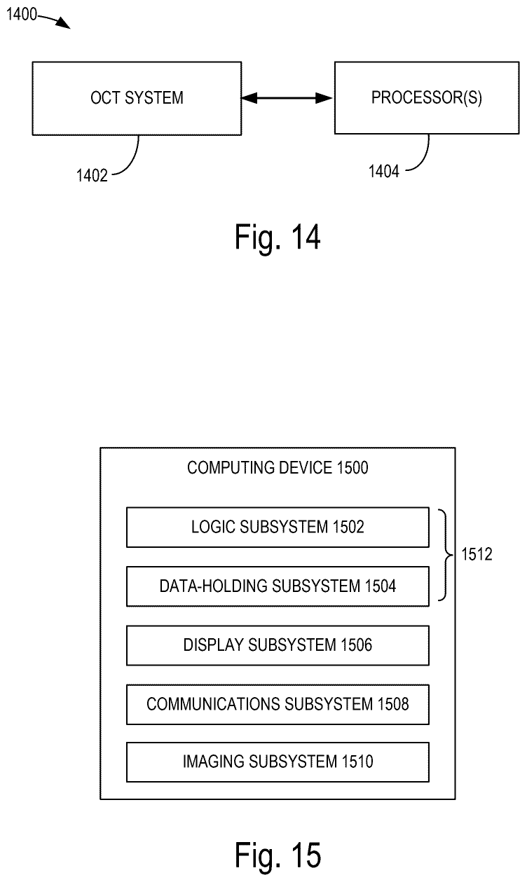

FIG. 14 depicts a block diagram of an example system 1400 for estimation of individualized baseline diagnostic maps and profiles, in accordance with various embodiments. The system is for OCT and OCT angiography image processing in accordance with various embodiments. System 1400 comprises an OCT system 1402 configured to acquire an OCT image comprising OCT interferograms and one or more processors or computing systems 1404 that are configured to implement the various processing routines described herein. OCT system 1400 may comprise an OCT system suitable for OCT imaging and/or OCT angiography applications, e.g., a swept source OCT system.

In various embodiments, an OCT system may be adapted to allow an operator to perform various tasks. For example, an OCT system may be adapted to allow an operator to configure and/or launch various ones of the herein described methods. In some embodiments, an OCT system may be adapted to generate, or cause to be generated, reports of various information including, for example, reports of the results of scans run on a sample.

In embodiments of OCT systems comprising a display device, data and/or other information may be displayed for an operator. In embodiments, a display device may be adapted to receive an input (e.g., by a touch screen, actuation of an icon, manipulation of an input device such as a joystick or knob, etc.) and the input may, in some cases, be communicated (actively and/or passively) to one or more processors. In various embodiments, data and/or information may be displayed, and an operator may input information in response thereto.

In some embodiments, the above-described methods and processes may be tied to a computing system, including one or more computers. In particular, the methods and processes described herein may be implemented as a computer application, computer service, computer API, computer library, and/or other computer program product.

FIG. 15 depicts a block diagram of an example computing system, in accordance with various embodiments. The system includes a non-limiting computing device 1500 that may perform one or more of the above described methods and processes. For example, computing device 1500 may represent a processor included in system 1400 described above, and may be operatively coupled to, in communication with, or included in an OCT system or OCT image acquisition apparatus. Computing device 1500 is shown in simplified form. It is to be understood that virtually any computer architecture may be used without departing from the scope of this disclosure. In different embodiments, computing device 1500 may take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 1500 includes a logic subsystem 1502 and a data-holding subsystem 1504. Computing device 1500 may optionally include a display subsystem 1506, a communication subsystem 1508, an imaging subsystem 1510, and/or other components not shown in FIG. 15. Computing device 1500 may also optionally include user input devices such as manually actuated buttons, switches, keyboards, mice, game controllers, cameras, microphones, and/or touch screens, for example.

Logic subsystem 1502 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. For example, the one or more processors may comprise physical circuitry programmed to perform various acts described herein. Additionally, or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 1504 may include one or more physical, non-transitory, devices, e.g., memory, configured to hold data and/or instructions executable by the logic subsystem to implement the herein described methods and processes. When such methods and processes are implemented, the state of data-holding subsystem 1504 may be transformed (e.g., to hold different data).

Data-holding subsystem 1504 may include removable media and/or built-in devices. Data-holding subsystem 1504 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1504 may include devices with one or more of the following characteristics: volatile, nonvolatile, dynamic, static, read/write, read-only, random access, sequential access, location addressable, file addressable, and content addressable. In some embodiments, logic subsystem 1502 and data-holding subsystem 1504 may be integrated into one or more common devices, such as an application specific integrated circuit or a system on a chip.

FIG. 15 also shows an aspect of the data-holding subsystem in the form of tangible, non-transitory computer-readable storage media 1512, which may be used to store and/or transfer data and/or instructions executable to implement the herein described methods and processes. The computer-readable storage media 1512 may take the form of CDs, DVDs, HD-DVDs, Blu-Ray Discs, EEPROMs, flash memory cards, USB storage devices, and/or floppy disks, among others.

When included, display subsystem 1506 may be used to present a visual representation of data held by data-holding subsystem 1504. As the described methods and processes change the data held by the data-holding subsystem, and thus transform the state of the data-holding subsystem, the state of display subsystem 1506 may likewise be transformed to visually represent changes in the underlying data. Display subsystem 1506 may include one or more display devices utilizing virtually any type of technology. Such display devices may be combined with logic subsystem 1502 and/or data-holding subsystem 1504 in a shared enclosure, or such display devices may be peripheral display devices.

When included, communication subsystem 1508 may be configured to communicatively couple computing device 1500 with one or more other computing devices. Communication subsystem 1508 may include wired and/or wireless communication devices compatible with one or more different communication protocols. As non-limiting examples, the communication subsystem may be configured for communication via a wireless telephone network, a wireless local area network, a wired local area network, a wireless wide area network, a wired wide area network, etc. In some embodiments, the communication subsystem may allow computing device 1500 to send and/or receive messages to and/or from other devices via a network such as the Internet.

When included, imaging subsystem 1510 may be used to acquire and/or process any suitable image data from various sensors or imaging devices in communication with computing device 1500. For example, imaging subsystem 1510 may be configured to acquire OCT image data, e.g., interferograms, as part of an OCT system, e.g., OCT system 1402 described above. Imaging subsystem 1510 may be combined with logic subsystem 1502 and/or data-holding subsystem 1504 in a shared enclosure, or such imaging subsystems may comprise periphery imaging devices. Data received from the imaging subsystem may be held by data-holding subsystem 1504 and/or removable computer-readable storage media 1512, for example.

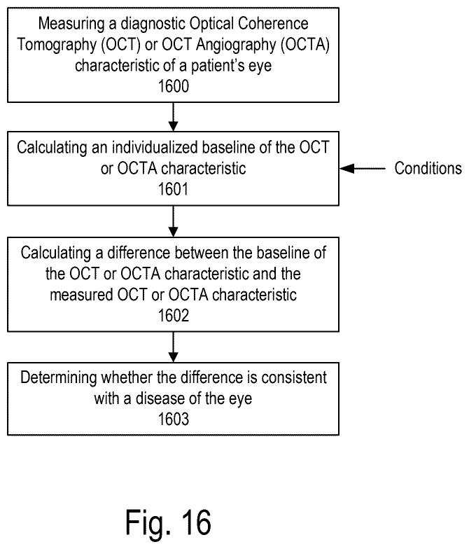

FIG. 16 depicts a flowchart of an example process for predicting a presence of disease in a patient's eye, in accordance with various embodiments. Step 1600 includes measuring the OCT or OCTA characteristic of an individual patient's eye. Step 1601 includes calculating an individualized baseline of the OCT or OCTA characteristic. This step can be a function of one or more conditions of the patient such as demographic information, one or more conditions regarding an anatomy of the eye such as disc area, axial length and spherical error and one or more conditions of the OCT or OCTA process such as a signal strength index. Step 1602 includes calculating a difference between the individualized baseline of the OCT or OCTA characteristic and the measured OCT or OCTA characteristic. Step 1603 includes determining whether the difference is consistent with a disease of the eye.

The following is list of acronyms used
herein: AI—artificial intelligence
AL—axial length
AROC—area under receiver
characteristic curve BASE—a deep
learning model
CEI—Casey Eye Institute
CNN—convolutional neural network
CVAE—conditional variational
autoencoder FDT—frequency doubling
technology
FPR—false positive rate
ILM—inner limiting
membrane KL—Kullback-Leibler
divergence List of
acronyms:
NFLT—nerve fiber layer
thickness MAG—a deep
learning model MLR—
multiple linear regression
MSE—mean square error
NFLT—nerve fiber layer
thickness OCT—Optical
coherence tomography ONH—
optic nerve head
PG—perimetric glaucoma
PPG—pre-perimetric glaucoma
rCNN—convolutional neural network for regression
analysis REG—a deep learning model
RNFLT—representative NFLT RPE—retinal pigment
epithelium SE—spherical equivalent
SSI—signal strength
index VAE—variational
autoencoder

Skilled persons will appreciate that many changes may be made to the details of the above-described embodiments without departing from the underlying principles of the invention. The scope of the present invention should, therefore, be determined only by claimed inventions and equivalents thereof.

What is claimed is:

1. A computer-implemented method, comprising:
measuring a diagnostic Optical Coherence Tomography (OCT) or OCT angiography (OCTA) characteristic of a patient's eye;
calculating an individualized baseline of the diagnostic OCT or OCTA characteristic based on an anatomic feature of the eye;
calculating a difference between the individualized baseline of the diagnostic OCT or OCTA characteristic and the measured OCT or OCTA characteristic; and
determining whether the difference is consistent with a disease of the eye.

2. The computer-implemented method of claim 1, wherein the anatomic feature of the eye comprises at least one of a vascular pattern, a retinal elevation, an optic disc size, or an optic disc shape.

3. The computer-implemented method of claim 1, wherein the calculating of the individualized baseline of the diagnostic OCT or OCTA characteristic is based on demographic information including at least one of age, gender, or race.

4. The computer-implemented method of claim 1, wherein the calculating of the individualized baseline of the diagnostic OCT or OCTA characteristic is based on a signal strength index.

5. The computer-implemented method of claim 1, wherein the diagnostic OCT or OCTA characteristic comprises at least one of a nerve fiber layer thickness, a disc rim width, a ganglion cell complex thickness, a ganglion cell inner plexiform layer thickness, a nerve fiber layer reflectance, a nerve fiber plexus capillary density, a superficial vessel complex vessel density, or a ganglion cell layer plexus vessel density.

6. The computer-implemented method of claim 1, wherein the calculating of the individualized baseline of the diagnostic OCT or OCTA characteristic is based on an axial length (AL) and a spherical equivalent (SE) refractive error of the eye.

7. The computer-implemented method of claim 1, wherein the calculating of the individualized baseline of the diagnostic OCT or OCTA characteristic is performed by a model comprising an encoder, followed by a latent mapper, followed by a decoder.

8. The computer-implemented method of claim 1, wherein the diagnostic OCT or OCTA characteristic comprises a nerve fiber layer thickness, and the disease is glaucoma.

9. The computer-implemented method of claim 1, wherein the disease comprises at least one of ocular or neurological diseases.

10. An apparatus, comprising:
a memory configured to store instructions; and
a processor coupled to the memory, wherein the processor is configured to execute the instructions to:
measure a diagnostic characteristic of a patient's eye based on optical coherence tomography (OCT) or OCT angiography (OCTA);
calculate a disease-free individualized baseline of the diagnostic characteristic of the eye based on an anatomic feature of the eye;
calculate a difference between the disease-free individualized baseline of the diagnostic characteristic and the measured diagnostic characteristic; and
determine whether the difference is consistent with a disease of the eye.

11. The apparatus of claim 10, wherein the calculating of the individualized baseline of the diagnostic characteristic is based on at least one of a vascular pattern, a retinal elevation, an optic disc size, or an optic disc shape.

12. The apparatus of claim 10, wherein the calculating of the individualized baseline of the diagnostic characteristic is based on a signal strength index.

13. The apparatus of claim 10, wherein the diagnostic characteristic comprises at least one of a nerve fiber layer thickness, a disc rim width, a ganglion cell complex thickness, a ganglion cell inner plexiform layer thickness, a nerve fiber layer reflectance, a nerve fiber plexus capillary density, a superficial vessel complex vessel density, or a ganglion cell layer plexus vessel density.

14. The apparatus of claim 10, wherein the anatomic feature of the eye comprises at least one of a vascular pattern, a retinal elevation, an optic disc size, or an optic disc shape.

15. A computer-implemented method, comprising:
training a model with a dataset from a population of healthy eyes, wherein the model comprises an encoder, followed by a latent mapper, followed by a decoder;
measuring an Optical Coherence Tomography (OCT) or OCT angiography (OCTA) characteristic of an individual patient's eye;
calculating, from the trained model, an individualized baseline of the OCT or OCTA characteristic of the individual patient's eye based on an anatomic feature of the eye;
calculating a difference between the individualized baseline of the OCT or OCTA characteristic and the measured OCT or OCTA characteristic of the individual patient's eye; and
determining whether the difference is above a threshold consistent with a disease of the individual patient's eye.

16. The computer-implemented method of claim 15, wherein the training of the model comprises concatenating one or more conditions with the encoder and the latent mapper.

17. The computer-implemented method of claim 16, wherein the one or more conditions comprise at least one of disc area, axial length and spherical error.

18. The computer-implemented method of claim 15, wherein the OCT or OCTA characteristic comprises at least one of a nerve fiber layer thickness, a disc rim width, a ganglion cell complex thickness, a ganglion cell inner plexiform layer thickness, a nerve fiber layer reflectance, a nerve fiber plexus capillary density, a superficial vessel complex vessel density, or a ganglion cell layer plexus vessel density.

19. The computer-implemented method of claim 15, wherein the disease comprises at least one of an optic neuropathy or multiple sclerosis.

20. The computer-implemented method of claim 15, wherein the anatomic feature of the eye comprises at least one of a vascular pattern, a retinal elevation, an optic disc size, or an optic disc shape.
